(19) United States
(12) Patent Application Publication
(10) Pub. No.: US 2023/0109240 A1
(43) Pub. Date: Apr. 6, 2023

(54) COMPOSITIONS AND METHODS FOR STYLING HAIR

(71) Applicant: L'OREAL, Paris (FR)

(72) Inventors: Stephanie LAGA, Helmetta, NJ (US); Rita CHOKSHI, Monroe Township, NJ (US); Heather LEE, Kinnelon, NJ (US)

(21) Appl. No.: 17/956,991

(22) Filed: Sep. 30, 2022

Related U.S. Application Data
(60) Provisional application No. 63/250,830, filed on Sep. 30, 2021.
(60) Provisional application No. 63/308,871, filed on Feb. 10, 2022.

(51) Int. Cl. A61K 8/24; A61K 8/73; A61Q 5/06; A61K 8/49

(52) U.S. Cl. CPC A61K 8/24; A61K 8/73; A61Q 5/06; A61K 8/4973

(57) ABSTRACT

The disclosure relates to compositions comprising at least one inorganic phosphate and at least one polysaccharide thickening agent. The compositions can be used for styling or shaping hair, and/or for providing manageability benefits to hair. The disclosure also relates to methods of using the compositions.

### CROSS REFERENCE TO RELATED APPLICATIONS

This application claims priority to U.S. Provisional Patent Application Nos. 63/250,830, filed Sep. 30, 2021, and 63/308,871, filed Feb. 10, 2022.

### TECHNICAL FIELD

The present disclosure relates to compositions comprising at least one inorganic phosphate and at least one polysaccharide thickening agent, and methods of using the compositions for styling hair.

### BACKGROUND

Curly hair usually appears shorter than its actual length. In the multicultural beauty market, there is a desire for products that can elongate hair curls and give more visual length to hair. In addition, reducing the curls of very curly hair may increase the manageability and ease of styling of such hair.

For curly hair consumers, points of concern include that their curly hair appears shorter than its actual length, and/or that the curls are not sufficiently defined. In the multicultural beauty market, there is a desire for products that elongate and/or define curls to showcase the natural curl pattern. Existing products on the market that aid in preventing curl shrinkage often use thick galenics with various butters or oils that can leave a greasy or oily feeling on hair. Products that provide hold, such as styling gels, can also give elongating effects, but these products typically flake on hair. Therefore, the use of these elongation products generally requires frequent hair washing, which is especially undesirable for consumers who have high curl patterns and prefer limited number of washdays per week and who desire to keep styles that last throughout the day or multiple days. In addition, traditional elongation of hair, including relaxing or straightening hair, often involves chemical treatment or high heat that may cause damage to the hair fibers and/or irritate the scalp.

As such, consumers desire new and improved styling compositions that can deliver visible and lasting elongation to hair curls and/or that provide visible and lasting curl definition and hold, particularly under humid conditions, and at the same time impart various additional advantageous properties to the hair such as moisture, softness, smoothness, good bounce, good shine, and/or frizz control.

It has now surprisingly been found that cosmetic compositions comprising at least one inorganic phosphate and at least one anionic or nonionic polysaccharide thickening agent are able to improve the elongation of hair curls and/or provide curl definition and hold, particularly under humid conditions, as well as provide other benefits such as hair moisture, softness, frizz control, and/or manageability benefits to the hair.

### SUMMARY

The present disclosure relates to compositions for styling hair, for example by improving the elongation of hair curls and/or providing curl definition and/or hold. The compositions are optionally leave-in hair styling compositions. The compositions comprise (a) at least one inorganic phosphate, (b) at least one anionic or nonionic polysaccharide thickening agent, and (c) at least one solvent. The compositions may optionally further comprise (d) at least one lactone; (e) at least one polyol; (f) at least one anionic surfactant; and/or (g) at least one fixing polymer.

In various embodiments, the compositions comprise (a) from about 0.1% to about 20% of at least one inorganic phosphate, (b) from about 0.1% to about 10% of at least one polysaccharide thickening agent chosen from anionic or nonionic polysaccharide thickening agents, and (c) at least one solvent, wherein all amounts are by weight, relative to the total weight of the composition.

The total amount of the inorganic phosphate(s) in the compositions may range, in various embodiments, from about 0.5% to about 15%, such as from about 0.75% to about 12%, about 1% to about 10%, about 1.25% to about 8%, about 1.5% to about 5%, about 1.75% to about 4%, or about 2% to about 3% by weight, relative to the total weight of the composition.

The at least one inorganic phosphate may, in certain embodiments, be chosen from saccharide tetrasodium etidronate, tetrasodium pyrophosphate, etidronic acid, sodium monophosphate, sodium hexametaphosphate, or combinations thereof. In certain embodiments, the at least one inorganic phosphate is a salt, such as a sodium, potassium, calcium or magnesium salt. In certain embodiments, the at least one inorganic phosphate is chosen from inorganic polyphosphates or a salt thereof, which in some embodiments may comprise sodium hexametaphosphate.

The total amount of the polysaccharide thickening agent may range, in various embodiments, from about 0.2% to about 8%, such as from about 0.3% to about 5%, about 0.4% to about 3%, or about 0.5% to about 2% by weight, relative to the total weight of the composition.

The at least one polysaccharide thickening agent may, in various embodiments, be chosen from hydroxypropyl guar, carrageenan, xanthan gum, cellulose gum, cyclodextrin, sclerotium gum, polyvinyl alcohol, or mixtures thereof. In some embodiments, the polysaccharide thickening agent comprises xanthan gum.

The compositions as described herein may have a ratio of polysaccharide thickening agent to inorganic phosphate ranging from about 2:1 to about 1:10, from about 1:1 to about 1:10, from about 1:1 to about 1:9, from about 1:1 to about 1:8, from about 1:1 to about 1:7, from about 1:1 to about 1:6, from about 1:1 to about 1:5, from about 1:1 to about 1:4, from about 1:1 to about 1:3, from about 1:2 to about 1:3, or from about 1:1 to about 1:2, or may have a ratio of polysaccharide thickening agent to inorganic phosphate of at least about 1:1 or at least about 1:2, such as about 1:1, about 1:2, about 1:3, about 1:4, or about 1:5. For example, the ratio of polysaccharide thickening agent to inorganic phosphate ranges from about 1:1 to about 1:5, in particular from about 1:2 to about 1:3.

The compositions described herein may further optionally comprise at least one lactone, such as, for example, at least one lactone chosen from α-lactones, β-lactones, γ-lactones, δ-lactones, and ϵ-lactones, or mixtures thereof. In certain embodiments, the compositions have a ratio of at least one lactone to at least one inorganic phosphate ranging from about 5:1 to about 1:1, from about 4:1 to about 1:1, from about 3:1 to about 1:1, or from about 2:1 to about 1:1.

When present, the at least one lactone may be included in any desired amount, for example ranging from about 0.1% to about 10%, such as from about 0.5% to about 8%, from about 0.75% to about 5%, or from about 1% to about 3% by weight, relative to the total weight of the composition.

The compositions described herein may further optionally comprise at least one polyol, which may, in certain embodiments, be chosen from C₂-C₁₆ polyols, such as diols and triols. For example, the at least one polyol may be chosen from ethylene glycol, propylene glycol, butylene glycol, hexylene glycol, pentylene glycol, diethylene glycol, dipropylene glycol, 1,3-propanediol, glycerin, ethylhexylglycerin, diglycerin, caprylyl glycol, polyethylene glycols, or mixtures thereof.

When present, the polyol may be included in any desired amount, for example ranging from about 0.1% to about 10% by weight, from about 0.5% to about 8%, from about 0.75% to about 5%, or from about 1% to about 3% by weight, relative to the total weight of the composition.

The compositions described herein may further optionally comprise at least one anionic surfactant, for example at least one anionic surfactant chosen from anionic sulfate surfactants, anionic non-sulfate surfactants, anionic carboxylate surfactants, salts thereof, or mixtures thereof. In a preferred embodiment, the composition comprises at least one anionic sulfate surfactant.

When present, the anionic surfactant may be included in any desired amount, for example ranging from about 0.1% to about 15%, from about 0.2% to about 10%, from about 0.3% to about 5%, from about 0.4% to about 3%, or from about 0.5% to about 2% by weight, relative to the total weight of the composition.

In various exemplary and non-limiting embodiments, the compositions comprise (a) from about 0.1% to about 20% by weight, such as from about 1% to about 10% or from about 1% to about 5% by weight, of at least one inorganic phosphate or a salt thereof, such as at least one inorganic polyphosphate, for example chosen from tetrasodium etidronate, tetrasodium pyrophosphate, etidronic acid, sodium hexametaphosphate, trimetaphosphate, or mixtures thereof; (b) from about 0.1% to about 10% by weight of, individually or in combination, at least one anionic polysaccharide thickening agent chosen from hydroxypropyl guar, carrageenan, xanthan gum, cellulose gum, or mixtures thereof, and/or at least one nonionic polysaccharide thickening agent chosen from cyclodextrin, sclerotium gum, polyvinyl alcohol, or combinations thereof; (c) at least one solvent; (d) optionally from about 0.01% to about 10% by weight of at least one lactone chosen from lactone chosen from α-lactones, β-lactones, γ-lactones, δ-lactones, and ϵ-lactones, or mixtures thereof; (e) from about 0.01% to about 10% by weight of at least one polyol; and (f) at least one anionic surfactant, wherein all amounts are by weight relative to the total weight of the composition. Optionally, the compositions may further comprise (g) at least one fixing polymer. In some embodiments, the ratio of polysaccharide thickening agent to inorganic phosphate ranges from about 1:1 to about 1:5, for example from about 1:2 to about 1:3, and the composition has a pH ranging from about 4 to about 8, from about 5 to about 7, or from about 5.5 to about 6.5.

In yet further exemplary and non-limiting embodiments, the compositions comprise (a) from about 1% A to about 10% by weight sodium hexametaphosphate; (b) from about 0.1° A to about 10% by weight, individually or together, of xanthan gum and/or hydroxypropyl guar; (c) at least one solvent; (d) optionally, from 0.01% to 5% by weight of at least one lactone; (d) from about 0.01% to about 5% by weight of at least one linear or branched C₂-C₁₆ polyol chosen from diols and triols; and (e) from about 0.1% to about 3% by weight of at least one anionic surfactant chosen from anionic sulfate or anionic sulfonate surfactants, wherein all amounts are by weight relative to the total weight of the composition. Optionally, the compositions may further comprise (g) at least one fixing polymer. In some embodiments, the ratio of polysaccharide thickening agent to sodium hexametaphosphate ranges from about 1:1 to about 1:5, such as from about 1:2 to about 1:3, and the composition has a pH ranging from about 4 to about 8, from about 5 to about 7, or from about 5.5 to about 6.5.

The disclosure further relates to methods of using the compositions, for example methods of styling hair such as methods of elongating curls, methods of lengthening the visual appearance of hair, methods of reducing shrinking of hair length, methods of maintaining elongation of curls, and/or methods of reducing frizz, even in conditions of high relative humidity, wherein the methods comprise applying the composition to wet, damp, or dry hair. Optionally, the compositions are left on the hair for a period of a few hours to a few days, for example until the hair is washed. The methods generally provide one or more styling benefits to the hair, such as curl elongation, curl definition, frizz control, smoothness, and/or softness.

### BRIEF DESCRIPTION OF THE FIGURES

The accompanying drawings, which are incorporated herein and constitute part of this specification, illustrate exemplary embodiments of the disclosure, and, together with the general description given above and the description provided herein, serve to explain features of the disclosure.

FIGS. 1A-1C show images of hair swatches treated with compositions according to the disclosure having an inorganic phosphate and various polysaccharide thickening agents, and hair swatches treated with comparative compositions.

FIGS. 2A-2B show images of hair swatches treated with compositions according to the disclosure having various ratios of polysaccharide thickening agent to inorganic phosphate, and hair swatches treated with comparative compositions.

FIGS. 3A-3H show images of hair swatches treated with compositions according to the disclosure having a polysaccharide thickening agent and various inorganic phosphates, and hair swatches treated with comparative compositions.

FIGS. 4A-4H show images of hair swatches treated with compositions according to the disclosure having an inorganic phosphate and polysaccharide thickening agent at various pH measurements, and hair swatches treated with comparative compositions.

It is to be understood that the foregoing and following descriptions are exemplary and explanatory only, and are not intended to be restrictive of any subject matter claimed.

### DETAILED DESCRIPTION

The disclosure relates to compositions for styling hair, as well as to methods of using the compositions.

### I. Compositions

In various embodiments, compositions according to the disclosure comprise (a) at least one inorganic phosphate; (b) at least one anionic or nonionic polysaccharide thickening agent; (c) at least one solvent; (d) optionally at least one lactone; (e) optionally at least one polyol; (f) optionally at least one anionic surfactant; and (g) optionally at least one fixing polymer.

Compositions according to the disclosure may improve hair curl elongation and/or persistence of hair curl elongation, may improve curl definition and/or curl hold, and/or may provide additional benefits such as softness, smoothness, and/or frizz control to the hair.

### Inorganic Phosphate

Compositions according to the disclosure comprise at least one inorganic phosphate. The at least one inorganic phosphate may, in various embodiments, optionally be in acid or salt form. In certain embodiments, the inorganic phosphate is chosen from inorganic polyphosphates. For example, the at least one inorganic polyphosphate may be chosen from inorganic polyphosphates with more than one phosphate moiety, e.g. inorganic polyphosphates with two or more phosphate moieties, inorganic polyphosphates with three or more phosphate moieties, inorganic polyphosphates with four or more phosphate moieties, inorganic polyphosphates with five or more phosphate moieties, inorganic polyphosphates with six or more phosphate moieties, and so on. If the inorganic phosphate is in salt form, it may, by way of non-limiting example, be a sodium salt, a potassium salt, a calcium salt, a magnesium salt, etc.

By way of non-limiting example only, the at least one inorganic phosphate can be chosen from tetrasodium etidronate, tetrapotassium editronate, tetrasodium pyrophosphate, etidronic acid, calcium etidronate, magnesium etidronate, disodium etidronate, calcium phosphate, calcium hexametaphosphate, sodium monophosphate, sodium or potassium tripolyphosphate, sodium tetraphosphate, sodium pentaphosphate, sodium trimetaphosphate, sodium hexametaphosphate, hydrates thereof, or combinations thereof.

In various exemplary embodiments, the at least one inorganic phosphate may be present in the compositions in an amount of about 0.1% or more by weight, such as about 0.5% or more, about 1% or more, or about 2% or more by weight, relative to the total weight of the composition. In other embodiments, at least one inorganic polyphosphate may be present in the compositions in an amount up to about 20%, such as up to about 15%, up to about 12%, up to about 10%, up to about 9%, up to about 8%, up to about 7%, up to about 6%, up to about 5%, up to about 4%, up to about 3%, up to about 2%, or up to about 1% by weight, relative to the total weight of the composition.

By way of non-limiting example, the at least one inorganic phosphate is present in the composition in a total amount ranging from about 0.1% to about 20%, for example, from about 0.1% to about 15%, from about 0.1% to about 12%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 20%, from about 0.5% to about 15%, from about 0.5% to about 12%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1.5%, from about 1% to about 20%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 2% to about 20%, from about 2% to about 15%, from about 2% to about 12%, from about 2% to about 10%, from about 2% to about 9%, from about 2% to about 8%, from about 2% to about 7%, from about 2% to about 6%, from about 2% to about 5%, from about 2% to about 4%, or from about 2% to about 3% by weight, relative to the total weight of the composition. In one exemplary and non-limiting embodiment, the at least one inorganic phosphate is present in an amount ranging from about 0.1% to about 10%, such as from about 0.5% to about 8% or from about 1% to about 5% by weight, relative to the total weight of the composition, including all ranges and subranges thereof.

In various embodiments, the at least one inorganic phosphate is chosen from inorganic polyphosphates, and is present in any of the above amounts, such as, for example, an amount ranging from about 0.1% to about 10%, such as from about 0.5% to about 8%, from about 1% to about 5%, or from about 1% to about 3% by weight, relative to the total weight of the composition, including all ranges and subranges thereof.

For example, in various embodiments, the composition comprises sodium hexametaphosphate, present in any of the above amounts, such as, for example, an amount ranging from 0.1% to about 10%, such as from about 0.5% to about 8%, from about 1% to about 5%, or from about 1% to about 3% by weight, relative to the total weight of the composition, including all ranges and subranges thereof.

### Polysaccharide Thickening Agents

Compositions according to the disclosure comprise at least one polysaccharide thickening agent. The polysaccharide thickening agent may be chosen from anionic polysaccharide thickening agents, nonionic polysaccharide thickening agents, or mixtures thereof. The polysaccharide thickening agents which can be used include, by way of example only, inulins, celluloses, starches, guar gums, xanthan gums, pullulan gums, alginate gums, agar-agar gums, carrageenan gums, gellan gums, gum arabics, xyloses, tragacanth gums, or derivatives of the foregoing. In further embodiments, cellobiose, maltodextrin, scleroglucan, chitosan, ulvan, fucoidan, alginate, pectin, or heparin. Mixtures of two or more of any of these may also be chosen.

Non-limiting examples of gums include acacia, agar, algin, alginic acid, ammonium alginate, amylopectin, calcium alginate, calcium carrageenan, carnitine, carrageenan, dextrin, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and biosacharide gum. Modified gums or derivatives of gums may also be used, such as, for example, deacylated gellan gum, welan gum, or hydroxypropylated guar gum, such as Jaguar HP 105 sold by Rhodia.

Non-limiting examples of celluloses include hydroxyalkylcelluloses, such as hydroxymethylcellulose, methylhydroxyethylcellulose, hydroxyethylcelluloses, hydroxypropylmethylcellulose, hydroxybutylcellulose, hydroxyethylmethylcellulose (also known as methyl hydroxyethylcellulose) or hydropropylcelluloses, which may or may not contain a fatty chain. One particularly suitable hydroxypropylmethylcellulose is Methocel F4M sold by Dow Chemicals (INCI name: hydroxypropylmethylcellulose). Celluloses modified with groups comprising one or more nonionic fatty chains that can be used include hydroxyethylcelluloses, such as nonionic hydroxyethylcelluloses, modified by groups comprising at least one fatty chain, such as alkyl, arylalkyl or alkylaryl groups, or their mixtures, and in which the alkyl groups are e.g. C8-C22 alkyl groups, such as the product NATROSOL™ Plus Grade 330 CS (C16 alkyls), sold by Aqualon, corresponding to the INCI name cetylhydroxyethylcellulose, or the product BERMOCOLL® EHM 100 sold by Berol Nobel, and those modified with alkylphenyl polyalkylene glycol ether groups, such as the product AMERCELL POLYMER® HM-1500 (nonylphenyl polyethylene glycol (15) ether) sold by Amerchol that corresponds to the INCI name nonoxynyl hydroxyethylcellulose. Other particular thickening agents suitable for use in the compositions described herein are hydroxypropyl guar gums.

Non-limiting examples of starches include modified starches, starch-based polymers, methylhydroxypropyl starch, potato starch, modified potato starch, wheat starch, rice starch, starch crosslinked with octenyl succinic anhydride, starch oxide, dialdehyde starch, dextrin, British gum, acetyl starch, starch phosphate, carboxymethyl starch, hydroxyethyl starch, and hydroxypropyl starch.

In some embodiments, the polysaccharide thickening agents are chosen from carrageenan, xanthan gum, cellulose, carboxymethylcellulose, hydroxyethylcellulose, hydroxypropylcellulose, hydroxypropylmethylcellulose, methylcellulose, starch, starch acetate, hydroxyethyl starch, hydroxypropyl starch, guar gum, carboxymethyl guar gum, carboxymethylhydroxypropylguar gum, hydroxyethyl guar gum, hydroxypropyl guar, xylose gum, cellobiose, dextrin, maltodextrin, scleroglucan, chitosan, ulvan, fucoidan, alginate, pectin, heparin, inulin, levan, or mixtures thereof. In further embodiments, the polysaccharide thickening agents are chosen from sclerotium gum, carrageenan, xanthan gum, cellulose gum, cyclodextrin, or mixtures thereof.

In various exemplary embodiments, the total amount of the at least one polysaccharide thickening agent may vary, or combinations thereof, typically ranges from about 0.1% to about 10%, including all subranges therebetween, such as from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, or from about 1% to about 2% by weight, relative to the total weight of the composition, including all ranges and subranges thereof.

In various embodiments, the compositions comprise from about 0.1% to about 5%, such as from about 0.5% to about 4%, from about 0.75% to about 3%, or from about 0.5% to about 2% of polysaccharide thickening agents, by weight, relative to the total weight of the composition. For example, the compositions may, in exemplary embodiments, comprise a total amount of polysaccharide thickening agents in any of the above ranges, for example ranging from about 0.1% to about 5%, from about 0.5% to about 4%, from about 0.75% to about 3%, or from about 0.5% to about 2% by weight, relative to the total weight of the composition, wherein the polysaccharide thickening agent comprises at least one polysaccharide thickening agent chosen from sclerotium gum, carrageenan, xanthan gum, cellulose gum, cyclodextrin, or mixtures thereof.

In some embodiments, it may be beneficial to choose total amounts of the at least one inorganic phosphate and at least one polysaccharide thickening agent to provide beneficial results to the hair. For example, the weight ratio of polysaccharide thickening agent to inorganic phosphate may be at least about 1:1 or at least about 1:2, such as, for example, a ratio ranging from about 2:1 to about 1:10, from about 1:1 to about 1:10, from about 1:1 to about 1:9, from about 1:1 to about 1:8, from about 1:1 to about 1:7, from about 1:1 to about 1:6, from about 1:1 to about 1:5, from about 1:1 to about 1:4, from about 1:1 to about 1:3, from about 1:2 to about 1:3, or from about 1:1 to about 1:2. In various embodiments, the composition may have a weight ratio of polysaccharide thickening agent to inorganic phosphate of about 1:1, about 1:2, about 1:3, about 1:4, or about 1:5.

In a particular embodiment, the weight ratio of polysaccharide thickening agent to inorganic phosphate may range from about 1:1 to about 1:5, such as from about 1:2 to about 1:3.

### Lactones

Compositions according to the disclosure optionally comprise at least one lactone. In certain embodiments, the at least one lactone may be chosen from cyclic ester compounds comprising a heterocyclic ring wherein the heterocyclic ring is oxygen, represented by the general formula (I):

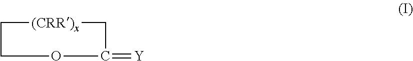

wherein:
R or R′, independently, is H, a hydrocarbon radical containing from 1 to 40 carbon atoms which may be saturated or unsaturated, linear or branched, substituted or unsubstituted, or a substituent other than H or said hydrocarbon radical, such as a hydroxy group, an amino group, a sulfhydryl group, an aryl group, or a halogen;
X represents an integer of 1 or more, such as an integer from between 1 to 10 or such as from 1 to 8 or such as from 1 to 6;
Y is oxygen (O) or sulfur (S); and
the heterocyclic ring is saturated or unsaturated.

For example, the lactone compounds of the present invention include, but are not limited, to those lactone compounds comprising 3- to 8-membered rings (including the oxygen on the heterocyclic ring and the carbonyl carbon).

Non-limiting examples of lactone compounds are α-lactones (3-membered ring alpha-lactones), β-lactones (4-membered ring beta-lactones), γ-lactones (5-membered ring gamma-lactones), δ-lactones (6-membered ring delta-lactones), and ϵ-lactones (8-membered ring epsilon-lactones).

In at least some embodiments, the lactone compounds of the present invention include, but are not limited to δ-lactones (delta-lactone) compounds corresponding to the formula (II):

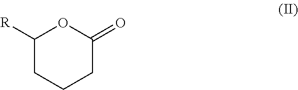

wherein:
R is chosen from hydrogen, a hydrocarbon radical containing from 1 to 40 carbon atoms, a hydroxyl group, an amino group, a sulfhydryl group, an aryl group, or a halogen;
the carbon atoms on the lactone ring, other than the R-substituted carbon atom and the carbon on the ketone group, may optionally be substituted; and
the heterocyclic ring is saturated or unsaturated,
with the proviso that when R is a hydrocarbon radical, R may be linear or branched, saturated or unsaturated, substituted or unsubstituted.

Non-limiting examples of δ-lactone compounds include delta-lactone, delta-octalactone, delta-decalactone, delta-nonalactone, undecanoic delta-lactone, delta-dodecalactone, massoia lactone (or 5-pentylpent-2-en-5-olide), jasmine lactone (or Z-2-pentenylpentan-5-olide), 6-Pentyl-alpha-pyrone (or 5-Pentylpenta-2,4-dien-5-olide) delta-valerolactone, galactonolactone, hexadecanolactone, and mevalonolactone.

Non-limiting examples of y-lactone compounds of the present invention are gamma valerolactone, gamma hexylactone, gamma hepatalactone, gamma otalactone, gamma nonalactone, gamma decalactone, gamma undecalactone and gamma dodecalactone.

Optionally, the at least one lactone compound based on formula (I) can be a thiocarbonyl analog of the lactone compound wherein Y in formula (I) is sulfur.

The at least one lactone compound of the present invention may also be chosen from lactone compounds wherein the heteroatom on the heterocyclic ring can be nitrogen or sulfur. When the heteroatom on the heterocyclic ring is nitrogen, the lactone compound may be chosen from lactam compounds and their thiocarbonyl analogs. When the heteroatom on the heterocyclic ring is sulfur, the lactone compound may be chosen from thiolactone compounds and their thiocarbonyl analogs.

One exemplary lactone compound of formula (I) is the δ-lactone compound Meadowfoam delta-lactone, commercially available from The Fanning Corporation under the tradename Meadowlactone, and corresponding to formula (III):

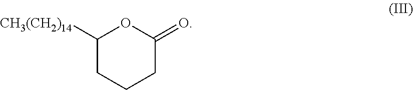

The at least one lactone compound may be present in an amount ranging from about 0.01% to about 10% by weight, such as from about 0.01% to about 8% by weight, from about 0.01% to about 6% by weight, from about 0.01% to about 4% by weight, from about 0.01% to about 2.5%, from about 0.01% to about 2%, from about 0.01% to about 1.5%, from about 0.1% to about 10%, from about 0.1% to about 8%, from about 0.1% to about 6%, from about 0.1% to about 4%, from about 0.1% to about 2.5%, from about 0.1% to about 2%, from about 0.1° A to about 1.5%, from about 0.5% to about 10%, from about 0.5% to about 8%, from about 0.5% to about 6%, from about 0.5% to about 4%, from about 0.5% to about 2.5%, from about 0.5% to about 2%, from about 0.5% to about 1.5%, from about 1% to about 10%, from about 1° A to about 8%, from about 1% to about 6%, from about 1% to about 4%, from about 1% to about 2.5%, or from about 1% to about 2% by weight, based on the total weight of the composition, including all ranges and subranges there between.

### Polyols

Compositions according to the disclosure optionally comprise at least one polyol. By way of example only, the at least one polyol may be chosen from C₂-C₁₆ polyols, such as C₂-C₁₂ or C₂-C₈ polyols. The at least one polyol may optionally be chosen from diols and triols. The at least one polyol may be linear or branched, saturated or unsaturated, and substituted or unsubstituted. Any stereoisomer of the polyols may also be used.

In various exemplary embodiments, the polyols may be chosen from glycols such as ethylene glycol, propylene glycol, butylene glycol, hexylene glycol, pentylene glycol, 1,3-propanediol, diethylene glycol, triethylene glycol, tetraethylene glycol, pentaethylene glycol, dipropylene glycol, caprylyl glycol, glycerin, diglycerin, ethylhexyglycerin, or mixtures thereof.

In other embodiments, the polyols may be chosen from, for example, propanediol, butanediol, pentanediol, hexanediol, heptanediol, octanediol, 1,2,6-hexanetriol, 1,2,4-butanetriol, trimethylolpropane, 2-butene-1,4-diol, 2-ethyl-1,3-hexanediol, 2-methyl-2,4-pentanediol, caprylyl glycol, 1,2-hexanediol, 1,2-pentanediol, 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, 2-ethyl-1,3-hexanediol, and 4-methyl-1,2-pentanediol.

In some embodiments, the compositions according to the disclosure comprise at least two polyols, for example at least two polyols chosen from diols and triols. As one non-limiting example, a composition according to the disclosure may comprise a combination of at least one glycerin compound and at least one additional glycol compound.

In various embodiments, the total amount of the at least one polyol can be about 1% or more, about 2% or more, about 3% or more, about 4% or more, about 5% or more, about 6% or more, or about 10% by weight, based on the total weight of the composition. In some embodiments, the at least one polyol is present in the composition with a total amount ranging from about 0.1% to about 10%, such as from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 8%, from about 0.5% to about 7%, from about 0.5% to about 6%, from about 0.5% to about 5%, from about 0.5% to about 4% by weight, from about 0.5% to about 3%, from about 0.5% to about 2%, or from about 0.5% to about 1.5% by weight, relative to the total weight of the composition.

In one exemplary and non-limiting embodiment, the at least one polyol is chosen from diols and triols, and is present in any of the above amounts, for example an amount of about 1%, about 1.5%, about 2%, about 3%, about 4% or about 5% or more by weight, relative to the total weight of the composition, and optionally comprises glycerin and/or at least one glycol. In a further exemplary and non-limiting embodiment, the at least one polyol is chosen from diols and triols, and is present in an amount ranging from about 0.5% to about 5%, such as from about 0.75% to about 4%, or about 1% to about 3% by weight, relative to the total weight of the composition, and comprises glycerin and/or at least one glycol.

### Anionic Surfactants

Compositions according to the disclosure optionally comprise at least one anionic surfactant. In various embodiments, suitable anionic surfactants may be chosen from sulfate/sulfonate, non-sulfate, or carboxylate anionic surfactants. For example alkyl sulfates, alkyl ether sulfates, alkyl monoglyceride sulfates, alkylaryl sulfates, alkyl ether carboxylates, fatty acyl sarcosinates, fatty acyl glutamates, alkyl phosphates, alkyl sulfoacetates, alkoxylated monoacids, alkyl sulfonates, alkyl amidesulfonates, alkylaryl sulfonates, alkyl monoglyceride sulfonates, α-olefin sulfonates, paraffin sulfonates, alkyl sulfosuccinates, alkyl ether sulfosuccinates, alkyl amidesulfosuccinates, alkyl sulfoacetates, N-acyltaurates, acylisethionates, alkyl sulfolaurates, salts thereof, or mixtures thereof may be chosen, wherein the alkyl groups of these compounds optionally including from 6 to 30 carbon atoms, such as from 12 to 28 carbon atoms, from 14 to 24 carbon atoms, or from 16 to 22 carbon atoms, wherein the aryl group of these compounds is optionally chosen from phenyl or benzyl groups, and wherein these compounds are optionally polyoxyalkylenated, for example polyoxyethylenated including from 1 to 50 ethylene oxide units such as from 2 to 10 ethylene oxide units. In certain preferred embodiments, the anionic surfactant comprises one or more alkyl sulfonates.

When the anionic surfactants are in salt form, they may be chosen from, for example, alkali metal salts such as the sodium or potassium salts, ammonium salts, amine salts such as the amino alcohol salts, or alkaline-earth metal salts such as the magnesium salt.

In various embodiments, the anionic surfactants may be chosen from, for example, C6-C24 alkyl sulfates, for example C12-C20 alkyl sulfates; C6-C24 alkyl ether sulfates, for example C12-C20 alkyl ether sulfates, e.g. comprising from 2 to 20 ethylene oxide units; C6-C24 alkyl sulfosuccinates, for example C12-C20 alkyl sulfosuccinates, e.g. lauryl sulfosuccinates; C6-C24 alkyl ether sulfosuccinates, for example C12-C20 alkyl ether sulfosuccinates; C6-C24 acylisethionates, for example C12-C18 acylisethionates; C6-C24 acylsarcosinates, for example C12-C20 acylsarcosinates, e.g. palmitoylsarcosinates; C6-C24 alkyl ether carboxylates, for example C12-C20 alkyl ether carboxylates; polyoxyalkylenated (C6-C24) alkyl(amido) ether carboxylic acids and salts thereof, e.g. those having from 2 to 50 alkylene oxide, and in particular ethylene oxide, groups; C6-C24 acylglutamates, for example C12-C20 acylglutamates; C6-C24 acylglycinates, for example C12-C20 acylglycinates; or mixtures thereof.

In at least certain embodiments, the compositions may comprise at least one anionic surfactant chosen from linear α-olefin sulfonates or salts thereof. For example, the compositions may comprise at least one anionic surfactant chosen from compounds of formula (IV) or salts thereof:

R—CH2—CH═CH—(CH2)n-SO3—-M (IV)

wherein:
R is a saturated linear alkyl radical comprising from 4 to 30 carbon atoms, such as from 6 to 20 carbon atoms or from 8 to 18 carbon atoms, such as from 10 to 14 carbon atoms;
n is an integer ranging from 0 to 10, such as from 1 and 4, optionally n=1; and
M is a cosmetically acceptable cation, for example cations derived from alkali metals or alkaline-earth metals or cations derived from organic amines such as alkanolamines, and may be, for example, chosen from ammonium, sodium, or potassium.

In certain embodiments, the at least one anionic surfactant is chosen from linear α-olefin sulfonates according to formula (V) or salts thereof:

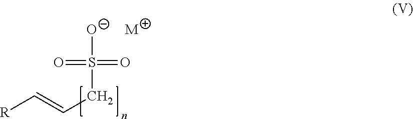

wherein R is an alkyl group, M is a sodium cation, and n is 1 or 2. For example, the at least one anionic surfactant may be sodium C14-C16 olefin sulfonate.

In various embodiments, if present, the total amount of the at least one anionic surfactant may range up to about 15% by weight, based on the total weight of the composition, including all ranges and subranges therebetween. For instance, the total amount of the at least one anionic surfactant may range from about 0.1% to about 15%, from about 0.1% to about 10%, from about 0.1% to about 5%, from about 0.1% to about 3%, from about 0.1% to about 2%, from about 0.5% to about 15%, from about 0.5% to about 10%, from about 0.5% to about 5%, from about 0.5% to about 3%, from about 0.5% to about 2%, from about 1% to about 15%, from about 1% to about 10%, from about 1% to about 5%, from about 1% to about 3%, or from about 1% to about 2% by weight, relative to the total weight of the composition.

In certain embodiments, the at least one anionic surfactant is chosen from anionic sulfate or sulfonate surfactants, for example alkyl sulfonate surfactants, such as linear a-olefin sulfonates, and is present in any of the above amounts, for example an amount ranging from about 0.01% to about 2.5% by weight, such as from about 0.1% to about 1% by weight, based on the total weight of the composition.

### Fixing Polymers

Compositions according to the disclosure optionally comprise at least one fixing polymer. In various embodiments, the fixing polymers are chosen from anionic fixing polymers, nonionic fixing polymers, amphoteric fixing polymers, or mixtures thereof.

### Anionic Fixing Polymers

Useful and non-limiting examples of anionic fixing polymers that may be used include polymers containing groups derived from carboxylic acid, sulfonic acid, or phosphoric acid, and having a number-average molecular weight ranging from about 500 to about 5,000,000.

By way of example only, anionic fixing polymers containing carboxylic acid derived groups that may be chosen include copolymers of acrylic or methacrylic acid or salts thereof, such as, for example, copolymers of acrylic or methacrylic acid with a monoethylenic monomer, such as ethylene, styrene, vinyl esters or acrylic or methacrylic acid esters, optionally grafted to a polyalkylene glycol, such as polyethylene glycol, and optionally crosslinked, the copolymers of this type optionally comprising an N-alkylated and/or hydroxyalkylated acrylamide unit in their chain. As further examples, the anionic fixing polymers may be chosen from copolymers of acrylic acid and of C₁-C₄ alkyl methacrylate and terpolymers of vinylpyrrolidone, of acrylic acid and of C₁-C₂₀ alkyl methacrylate, for example lauryl methacrylate, such as that sold by ISP under the name Acrylidone® LM (INCI name: VP/acrylates/lauryl methacrylate copolymer), acrylic acid/ethyl acrylate/N-(t-butyl)acrylamide terpolymers, such as the products Ultrahold® Strong and Ultrahold® 8 sold by BASF (INCI name: Acrylates/t-butylacrylamide copolymer), methacrylic acid/ethyl acrylate/tert-butyl acrylate terpolymers, such as the products sold under the names Luvimer® 100 P or Luvimer® PRO 55 by BASF (INCI name: Acrylates copolymer), copolymers of methacrylic acid and of ethyl acrylate, such as the products sold under the names Luvimer® MAE or Luviflex® Soft by BASF (INCI name: Acrylates copolymer), acrylic acid/butyl acrylate/methyl methacrylate terpolymers, such as the product sold under the name Balance® CR by AkzoNobel (INCI name: Acrylates copolymer), or the copolymers of methacrylic acid and of methyl methacrylate sold under the name Eudragit® L 100 by Rohm Pharma (INCI name: Acrylates copolymer).

As further examples, crotonic acid copolymers, such as those comprising vinyl acetate or propionate units in their chain and optionally other monomers such as allyl esters or methallyl esters, vinyl ether, or vinyl ester of a linear or branched saturated carboxylic acid with a long hydrocarbon-based chain, such as those containing at least 5 carbon atoms, optionally grafted or crosslinked, or alternatively another vinyl, allyl or methallyl ester monomer of an α- or β-cyclic carboxylic acid, such as Resyn® 28-2930 (INCI name VA/crotonates/vinyl decanoate copolymer) and 28-1310 (INCI name VA/crotonates copolymer) sold by the company AkzoNobel, may be chosen.

As still further examples of anionic fixing polymers containing carboxylic acid derived groups that may be chosen, mention may be made of copolymers of C₄-C₈ monounsaturated carboxylic acids or anhydrides chosen from copolymers comprising (i) one or more maleic, fumaric or itaconic acids or anhydrides and (ii) at least one monomer chosen from vinyl esters, vinyl ethers, vinyl halides, phenylvinyl derivatives, or acrylic acid and its esters, or copolymers comprising (i) one or more maleic, citraconic or itaconic anhydride units and (ii) one or more monomers chosen from allyl or methallyl esters, optionally comprising one or more acrylamide, methacrylamide, α-olefin, acrylic or methacrylic ester, acrylic or methacrylic acid or vinylpyrrolidone groups in their chain, the anhydride functions of these copolymers optionally being monoesterified or monoamidated.

As exemplary and non-limiting anionic fixing polymers containing sulfonic acid derived groups, homopolymers and copolymers comprising vinylsulfonic, styrenesulfonic, naphthalenesulfonic, or acrylamidoalkylsulfonic units may be chosen. As further examples, linear or branched sulfonic polyesters, for example obtained by polycondensation of at least one dicarboxylic acid, of at least one diol, or of a mixture of diol and of diamine, and of at least one difunctional monomer comprising a sulfonic function, for example the products Eastman® AQ38S Polymer, Eastman® AQ55S Polymer and Eastman® AQ48 Ultra Polymer sold by the company Eastman Chemical (Polyester-5) which are copolymers obtained from diethylene glycol, from 1,4-cyclohexanedimethanol, from isophthalic acid, and from sulfoisophthalic acid salt, or Eastman® AQ10D Polymer (Polyester-13), may be chosen.

### Nonionic Fixing Polymers

Useful and non-limiting examples of nonionic fixing polymers that may be chosen include polyalkyloxazolines; vinyl acetate homopolymers, vinyl acetate copolymers, such as, for example, copolymers of vinyl acetate and of acrylic ester, copolymers of vinyl acetate and of ethylene, or copolymers of vinyl acetate and of maleic ester, for example of dibutyl maleate; acrylic ester homopolymers and copolymers, such as, for example, copolymers of alkyl acrylates and of alkyl methacrylates, such as the products provided by Rohm GmbH under the name Eudragit® NE 30 D (INCI name: Acrylates copolymer); copolymers of acrylonitrile and of a nonionic monomer chosen, for example, from butadiene and alkyl (meth)acrylates; styrene homopolymers; styrene copolymers, such as, for example, copolymers of styrene, of alkyl acrylate and of alkyl methacrylate; copolymers of styrene and of butadiene; or copolymers of styrene, of butadiene and of vinylpyridine; polyamides; vinyllactam homopolymers, such as the vinylpyrrolidone homopolymers sold, for example, under the names Luviskol® K30 powder by the company BASF or PVP K30L or K60 solution or K90 by the company ISP, or such as the polyvinylcaprolactam sold under the name Luviskol® Plus by the company BASF (INCI name PVP); vinyllactam copolymers, such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVP/VA® S630L, E735, E635 and W735 by the company ISP, Luviskol® VA 73, VA 64 and VA 37 by the company BASF (INCI name VP/VA copolymer); and vinylpyrrolidone/methacrylamide/vinylimidazole terpolymers, for instance the product sold under the name Luviset® Clear by the company BASF (INCI name VP/methacrylamide/vinyl imidazole copolymer). In various embodiments, the alkyl groups of these polymers optionally contain from 1 to 6 carbon atoms.

In certain embodiments, the nonionic fixing polymer is chosen from vinyllactam homopolymers such as homopolymers of vinylpyrrolidone and polyvinylcaprolactam, vinyllactam copolymers such as poly(vinylpyrrolidone/vinyllactam) copolymers and poly(vinylpyrrolidone/vinyl acetate) copolymers, or mixtures thereof.

### Amphoteric Fixing Polymers

The amphoteric fixing polymers which can be used include, for example, polymers comprising B and C units randomly distributed in the polymer chain, where B denotes a unit deriving from a monomer comprising at least one basic nitrogen atom and C denotes a unit deriving from an acidic monomer comprising one or more carboxylic or sulfonic groups, or else B and C can denote groups deriving from zwitterionic carboxybetaine or sulfobetaine monomers, B and C may also denote a cationic polymer chain comprising primary, secondary, tertiary, or quaternary amine groups, in which at least one of the amine groups bears a carboxylic or sulfonic group connected via a hydrocarbon-based group, or alternatively B and C form part of a chain of a polymer comprising an α,β-dicarboxylic ethylene unit in which one of the carboxylic groups has been made to react with a polyamine comprising one or more primary or secondary amine groups.

By way of non-limiting example, amphoteric polymers may be chosen from copolymers having acidic vinyl units and basic vinyl units, such as those resulting from the copolymerization of a monomer derived from a vinyl compound bearing a carboxylic group such as, more particularly, acrylic acid, methacrylic acid, maleic acid, alpha-chloroacrylic acid, and a basic monomer derived from a substituted vinyl compound containing at least one basic atom, such as, for example, dialkylaminoalkyl methacrylate and acrylate, or dialkylaminoalkylmethacrylamides and acrylamides. As further examples, polymers comprising units deriving from at least one monomer chosen from acrylam ides and methacrylam ides substituted on the nitrogen atom with an alkyl group, for example N-ethylacrylamide, N-tert-butylacrylamide, N-tert-octylacrylamide, N-octylacrylamide, N-decylacrylamide, N-dodecylacrylamide and the corresponding methacrylamides; from at least one acidic comonomer containing one or more reactive carboxylic groups, such as acrylic acid, methacrylic acid, crotonic acid, itaconic acid, maleic acid, or fumaric acid; or from at least one basic comonomer such as esters with primary, secondary, tertiary and quaternary amine substituents of acrylic and methacrylic acids and the product of quaternization of dimethylaminoethyl methacrylate with dimethyl or diethyl sulfate, for example aminoethyl, butylaminoethyl, N,N′-dimethylaminoethyl, or N-tert-butylaminoethyl methacrylates, may be chosen.

As further non-limiting examples of amphoteric fixing polymers, methyl methacrylate/methyl dimethylcarboxymethylammonioethyl methacrylate copolymers, such as the product sold under the name Diaformer Z-301N or Z-301W by the company Clariant (INCI name Acrylates copolymer), polymers derived from chitosan, polymers derived from the N-carboxyalkylation of chitosan, such as N-(carboxymethyl)chitosan or N-(carboxybutyl)chitosan, such as, for example, the product sold under the name Chitoglycan by Sinerga SPA (INCI name: Carboxymethyl chitosan), or (C₁-C₅)alkyl vinyl ether/maleic anhydride copolymers, partially modified by semiamidation with an N,N-dialkylaminoalkylamine, such as N,N-dimethylaminopropylamine, or by semiesterification with an N,N-dialkylaminoalkanol may be used. In some embodiments, Octylacrylamide/acrylates/butylaminoethyl methacrylate copolymer, such as the products sold under the names Amphomer®, Amphomer® LV 71, or Balance® 47 by the company AkzoNobel, or methyl methacrylate/methyl dimethyl-carboxymethylammonioethyl methacrylate copolymers, sold, for example, under the name Diaformer Z-301N or Z-301W by the company Clariant, may be particularly useful.

If present, the at least one fixing polymer may be present in an amount ranging from about 0.1% to about 10% by weight, such as from about 0.1% to about 8%, about 0.1% to about 6%, about 0.1% to about 5%, about 0.1% to about 4%, about 0.1% to about 3%, about 0.1% to about 2%, about 0.1% to about 1%, about 0.5% to about 10%, about 0.5% to about 8%, about 0.5% to about 6%, about 0.5% to about 5%, about 0.5% to about 4%, about 0.5% to about 3%, about 0.5% to about 2%, or about 0.5% to about 1% by weight, relative to the total weight of the composition.

In one exemplary embodiment, the compositions comprise at least one non-ionic fixing polymer, present in any of the above amounts, for example an amount ranging from about 0.1% to about 5% by weight, such as from about 0.5% to about 4% by weight, or about 0.5% to about 3% by weight, relative to the total weight of the composition.

### Solvents

Compositions according to the disclosure comprise a cosmetically acceptable solvent. The solvent may comprise water, non-aqueous solvents, or mixtures thereof.

In some embodiments, the solvent comprises, consists essentially of, or consists of water. The total amount of water in the compositions may vary depending on the type of composition and the desired consistency, viscosity, etc.

In certain embodiments, the composition comprises one or more non-aqueous solvents, other than or in addition to ingredients discussed above. For example, C₁₋₄ alcohols, organic solvents, fatty alcohols, fatty ethers, fatty esters, polyols other than those described above, glycols, vegetable oils, mineral oils, liposomes, laminar lipid materials, or any a mixture thereof. Non-limiting examples of solvents which may be used include alkane polyols such as 1,2,6-hexanetriol, trimethylolpropane, ethylene glycol, propylene glycol, diethylene glycol, hexylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol (isopropyl alcohol); glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbitol, sorbitan, acetine, diacetine, triacetine, sulfolane, or mixtures thereof. At least in some embodiments, the compositions contain water and at least one additional solvent chosen from hexylene glycol, ethylhexylglycerin, or glycerin.

The solvent may be present in the composition in an amount ranging from about 50% to about 98% by weight, relative to the total weight of the composition, including all ranges and subranges therebetween. For example, in some embodiments, the total amount of solvent may be about 50% to about 95%, about 60% to about 95%, about 70% to about 95%, about 70% to about 90%, about 75% to 95%, or about 75% to 90% by weight, relative to the total weight of the composition. In certain embodiments, the solvent is primarily comprised of water, such as from about 90% to about 99%, or about 95% to about 99%, of the total solvent.

### Additional Components

Compositions according to the disclosure may optionally comprise any additional or auxiliary component suitable for use in such compositions. Such components may include, but are not limited to, conditioning agents, dyes/pigments, moisturizing agents, fillers, structuring agents, shine agents, antioxidants, penetrants, sequestrants, fragrances, buffers (e.g. monoethanolamine and/or triethanolamine), dispersants, plant extracts, plant oils, preserving agents (preservatives), opacifiers, sunscreen agents, pH adjusting agents, vitamins (e.g. Vitamin E), surfactants other than anionic surfactants, e.g. nonionic surfactants and/or amphoteric surfactants, and antistatic agents.

Optional additional or auxiliary components may be present in an amount ranging up to about 15% by weight, such as from about 0.01% to about 15% or about 0.1° A to about 10% by weight, relative to the total weight of the composition.

In various embodiments, the compositions may have a pH less than or equal to about 12.5. For example, the pH of the compositions may range from about 4 to about 12.5, such as from about 4.5 to about 12, from about 4.5 to about 9, from about 4.75 to about 8.52, from about 5.5 to about 6.5, including all ranges and subranges therebetween.

Compositions according to the disclosure are typically in the form of a liquid, cream, paste, gel, foam, such as mousse, or lotion, but are not limited to these forms. The term “gel” is generally understood to mean a soft, semi-solid, or solid-like material that exhibits low to no flow under steady state. In some embodiments, the compositions may be formulated as aerosol type products. In some embodiments, the compositions are non-aerosol type product. When the compositions are formulated as aerosol type products, the compositions may further comprise a propellant.

### II. Methods

The present disclosure also relates to methods for styling hair, for example for improving elongation and/or curl definition of curly hair by the use of the compositions described herein. The methods generally comprise applying any of the compositions according to the disclosure to the hair.

Compositions according to the disclosure are typically used for providing a visible elongation benefit to wavy or curly hair, for improving the elongation of hair waves or curls, for providing long lasting elongation of hair waves or curls (even at high humidity conditions and/or over a period of time), for improving curl definition, for providing long lasting curl definition of wavy or curly hair, and/or for reducing frizziness of hair. The compositions may be useful in a variety of settings, either for chemically treated or untreated hair, and/or for natural or unnatural (e.g. permed hair) waves or curls. The compositions described herein are particularly suited to multicultural hair types of varying curliness and hair types. The degree of curliness or curl type of the hair may vary and is not limited. For example, the curls of hair been treated by the compositions disclosed herein may range from slightly wavy to very kinky and coily hair, and may have different textures and colors.

In some embodiments, the compositions described herein are useful for increasing the perceived length of hair, reducing shrinkage of curly hair, and/or reducing frizziness of hair, particularly in humid and high humidity (e.g. 80% RH, 25° C.) conditions, by providing lasting curl elongation and/or curl definition of the hair, e.g. providing lasting curl elongation and/or curl definition for 1 hour or more, for 4 hours or more, for 8 hours or more, for 12 hours or more, for 16 hours or more, for 24 hours or more, or for 72 hours or more, after application of a composition according to the disclosure.

In other embodiments, the compositions are useful for improving definition of curls and/or curl hold, and/or reducing frizziness of hair, particularly in humid and high humidity (e.g. 80% RH, 25° C.) conditions, by providing lasting curl elongation and/or curl definition of the hair, e.g. providing lasting curl elongation and/or curl definition for 1 hour or more, for 4 hours or more, for 8 hours or more, for 12 hours or more, for 16 hours or more, for 24 hours or more, or for 72 hours or more, after application of a composition according to the disclosure.

In addition, the compositions may also be used to provide a variety of desirable sensory benefits, for example, moisture, smoothness, softness, good bounce, and/or good shine, to the hair. As such, the compositions are useful in styling (which includes shaping) hair while also caring for hair, conditioning hair, and/or imparting one or more above described sensory benefits to the hair.

Typically, methods according to the disclosure may include applying an effective amount of a composition disclosed herein to hair having curls, when the hair is dry, wet, damp, or moist. As used herein, the term “effective amount” refers to an amount sufficient to provide a desired elongation effect to the hair, taking into account the degree of curliness, the length, the volume, and the texture of the hair. In general, from about 0.5 grams to about 50 grams of product is applied to the hair, depending on the specific product formulation, hair length, hair volume, and hair style type. The composition applied to the hair may be distributed evenly by combing through with fingers or a means such as a comb or the like. The composition may then be dried, for example air-dried. The composition may be allowed to remain on the hair as a leave-in product for any period of time as needed, for example, from about a few seconds (e.g. 1, 3, 5, or 10 seconds) to about 10, 20, or 30 minutes, or longer, such as a few hours or a few days, or until the next washing or rinsing of the hair.

In some embodiments, before applying the composition to the hair, the hair may be first cleansed with a commercially available shampoo, or rinsed with water. The composition is then applied to the washed or rinsed hair when the hair is wet, damp, or moist. In some other embodiments, before applying the composition to dry hair, the hair can optionally be moistened, damped, or wetted by water spray or using a wet towel, or by applying other treatment compositions that make the hair moist, damp, or wet.

Alternatively, in some embodiments, the compositions according to the disclosure may be first applied to dry hair, and then water or a cosmetically acceptable compositions are applied to the hair to moist, damp, or wet the hair when the compositions according to the disclosure are remained thereon. The hair that has been applied with the compositions disclose herein may be air-dried without further treatment of the hair, including rinsing or washing the hair.

In some embodiments, the methods may optionally include braiding and/or twisting out the hair to make curls, before or after application of the compositions disclosed herein. In some embodiments, methods according to the disclosure do not require shaping or styling the hair before, during, and/or after applying the compositions. A tension to the hair is generally not needed. As such, in some embodiments, before, during, or after applying the compositions, the hair is not stretched by a mechanical means.

In some embodiments, after application of the compositions disclosed herein, the hair may be shaped or styled as needed, such as be twisted or the like. In some embodiments, the hair treated with the compositions may be twisted within 30 minutes, or within 20 minutes, while the hair is wet or damp.

Methods according to the disclosure generally do not require the use of a reducing agent, including a base, or heating the hair for stretching the hair curls or elongating the hair curls. As such, in at least some embodiments, the method does not including using a reducing agent, or a base, and/or does not include heating the hair.

In some embodiments, a composition disclosed herein is used alone to treat the hair, without the use of other hair treatment compositions. In some other embodiments, the compositions may be applied to the hair before, during, or after another hair treatment or styling composition, such as (e.g. a conditioner, a mask, a cream, a lotion, a gel, a coloring composition, etc.), is applied to the hair. As such, a composition disclosed herein may be applied to the hair and layered with another composition that is also applied to the hair.

In various embodiments, the methods of treating hair with the compositions according to the disclosure, impart various long-lasting benefits described above to the hair, without a greasy feel or flaking, relative to hair not having been treated with a composition according to the disclosure. The term “long-lasting,” as used herein, means that the elongation effect and other benefits imparted to the hair may remain a desired length of time, such as a few hours, a few days, or until the hair is rinsed or washed.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

The term “and/or” should be understood to include both the conjunctive and the disjunctive. For example, “A and/or B” means “A and B” as well as “A or B,” and expressly covers instances of either.

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1. In addition, endpoints of any range disclosed herein is expressly intended to be included in said range.

As used herein, if a component is described as being present “in an amount up to” a certain amount, it is intended that such component is, in fact, present in the composition, i.e. is present in an amount greater than 0%.

“Active material” as used herein with respect to the percent amount of an ingredient or raw material, refers to 100% activity of the ingredient or raw material.

All amounts given herein are relative to the amount of active material, unless otherwise indicated.

All percentages, parts and ratios herein are based upon the total weight of the compositions of the present disclosure, unless otherwise indicated.

As used herein, the term “applying a composition onto hair” and variations thereof means contacting the hair with at least one of the compositions of the disclosure, in any manner. It may also mean contacting the hair in an effective amount.

As used herein, the term “styling” the hair is intended to include “shaping,” the hair or altering the shape of hair, such as, for example, elongating hair curls or providing curl definition.

As used herein, the term “curly hair” refers to any hair including a curl or wave. The curl may be natural or unnatural, i.e., formed by chemical treatment or physical treatment of the hair. The degree of curliness of the hair may vary and is not limited.

As used herein, hair with improved or enhanced curl definition may have curls with a shape that has a clean ringlet appearance rather than being frizzy, curls that appear more individualized, curls that are more closed in appearance, and/or curls that have an improved visual appearance of the hair color and/or highlights.

As used herein, the terms “elongating” and “stretching” hair are interchangeable, and both refer to reducing the degree of waviness or curliness and/or tightness of waves or curls of the hair, and “improving the elongation of” hair or hair curls, and variants thereof, means that the length of the hair appears closer to the actual, uncurled length of the hair.

A “leave-in” composition or product refers to a composition such as a hair-treatment composition that is not rinsed and/or washed away with water or acceptable solvent after the application of the composition onto the hair; instead, the composition is allowed to remain on the hair for a period of time as desired, such from 1 hour, 2 hours, 3 hours, 4 hours, up to 8 hours, overnight, or as long as needed, until next time of washing or rinsing the hair.

As used herein, the term “inorganic” means a material that does not comprise carbon.

As used herein, the terms “inorganic phosphate” and “inorganic polyphosphates” are intended to include acid and/or salt forms of the compounds, as well as complexes.

As used herein, the term “high humidity” refers to conditions having at least 70% relative humidity or greater, such as at least 75% relative humidity or greater, or at least 80% relative humidity or greater.

As used herein, the term “organic” means a material that is produced substantially without or essentially without the use of synthetic materials. “Organic materials” include materials made of organic compounds/molecules comprising at least one carbon atom.

As used herein, the term “polyol” refers to an organic molecule comprising more than one free hydroxyl group.

The terms “substantially without” or “essentially without” as used herein means the specific material may be used in a manufacturing process in small amounts that do not materially affect the basic and novel characteristics of the compositions according to the disclosure. The terms may also mean that the specific material is not used in a manufacturing process but may still be present in a raw material that is included in the composition.

As used herein, the term “salts” throughout the disclosure may include salts having a counter-ion such as an alkali metal, alkaline earth metal, or ammonium counterion. This list of counterions, however, is non-limiting.

As used herein, the term “substantially free” or “essentially free” means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “surfactants,” as well as any specifically-identified surfactants, includes salts of the surfactants even if not explicitly stated.

As used herein, the term “synthetic” means a material that is not of natural origin. The term “natural” and “naturally-sourced” means a material of natural origin, such as derived from plants, which also cannot be subsequently chemically or physically modified. “Plant-based” means that the material came from a plant.

As used herein, the term “treat” (and its grammatical variations) refers to the application of the compositions of the present disclosure onto the surface of keratin materials, such as hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts are expressed in percentage by weight (wt %) of active materials, relative to the total weight of the composition.

### Example 1 — Compositions With Various Polysaccharide Thickening Agents

The inventive and comparative compositions in Table 1 were prepared, having various anionic and nonionic polysaccharide thickening agents.

TABLE 1

| | Inventive | | | | | Comparative | | | | | | |
|---|---|---|---|---|---|---|---|---|---|---|---|---|
| | 1A | 1B | 1C | 1D | 1E | C1 | C2 | C3 | C4 | C5 | C6 | C7 |
| Cyclodextrin | 1 | | | | | | 1 | | | | | |
| Sclerotium gum | | 1 | | | | | | 1 | | | | |
| Carrageenan | | | 1 | | | | | | 1 | | | |
| Xanthan gum | | | | 1 | | | | | | 1 | | |
| Cellulose gum | | | | | 1 | | | | | | 1 | |
| Sodium hexa- | 3 | 3 | 3 | 3 | 3 | 3 | | | | | | |
| metaphosphate | | | | | | | | | | | | |
| Water | QS to | QS to | QS to | QS to | QS to | QS to | QS to | QS to | QS to | QS to | QS to | QS to |
| | 100 | 100 | 100 | 100 | 100 | 100 | 100 | 100 | 100 | 100 | 100 | 100 |

Twelve swatches of curly hair were washed with a standard shampoo and rinsed, and then equal amounts of compositions 1A-1E or C1-C7 were applied to separate wet hair swatches. The treated hair swatches were then brushed 5 times and allowed to air dry for 24 hours. The film on the hair was subsequently broken and the swatches were evaluated for elongation and level of frizz. The swatches were then put into a humidity chamber at 80% relative humidity at 25° C. and again evaluated for elongation and frizz.

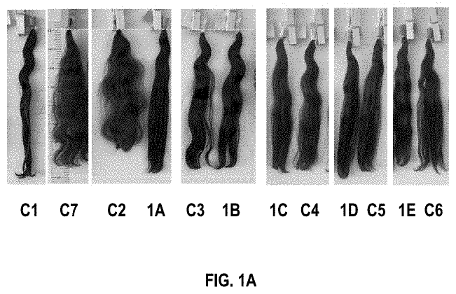

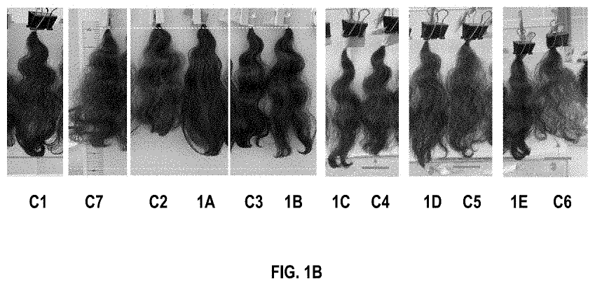

The hair swatches treated with each of compositions 1A-1E and C1-C7 were evaluated for elongation and frizz once the film was broken after drying for 24 hours (FIG. 1A), and again after 1 hour in the humidity chamber (FIG. 1B). In addition, hair swatches treated with each of compositions 1A, 1B, C2, C3, and C7 were evaluated for elongation and frizz after 8 hours in the humidity chamber (FIG. 1C).

As FIGS. 1A-1C show, hair treated with the combination of inorganic phosphate and polysaccharide thickening agent (compositions 1A-1E) demonstrate better elongation and frizz control than the comparative compositions comprising either an inorganic phosphate alone, polysaccharide thickening agent alone, or water alone (compositions C1-C7).

This Example demonstrates the surprising and unexpected synergistic benefits achieved with compositions comprising combinations of inorganic phosphates and various anionic and nonionic polysaccharide thickening agents according to the disclosure.

### Example 2—Compositions With Various Ratios

The inventive and comparative compositions in Table 2A were prepared, with the inventive compositions having varying ratios of polysaccharide thickening agent to inorganic phosphate (polysaccharide thickening agent:inorganic phosphate).

TABLE 2A

| | Inventive | | | | | | Comparative | |
|---|---|---|---|---|---|---|---|---|
| | 2A | 2B | 2C | 2D | 2E | 2F | C8 | C9 |
| Xanthan gum | 1 | 1 | 1 | 1 | 1 | 1 | 1 | |
| Sodium hexa- | 0.5 | 1 | 2 | 3 | 5 | 10 | | |
| metaphosphate | | | | | | | | |
| Water | QS to | QS to | QS to | QS to | QS to | QS to | QS to | QS to |
| | 100 | 100 | 100 | 100 | 100 | 100 | 100 | 100 |

### Example 2-1—Curly Hair

Eight swatches of curly hair were washed with a standard shampoo and rinsed, and then equal amounts of compositions 2A-2F or C8-C9 were applied to separate wet hair swatches. The treated hair swatches were then brushed 5 times and allowed to air dry for 24 hours (Time A). The film on the hair was subsequently broken and the swatches were evaluated for elongation and level of frizz. The swatches were then put into a humidity chamber at 80% relative humidity at 25° C. and again evaluated for elongation and frizz after 8 hours (Time B).

The elongation, frizz, and stiffness of each swatch was assigned a rating from 1 to 5, with a rating of 1 meaning that the swatch had the most frizz, least elongation, and was the least stiff, and a rating of 5 meaning that the swatch had the least frizz, most elongation, and was the most stiff. Such an evaluation is how properties imparted by such compositions are typically evaluated in the art, since beneficial properties of new formulations generally manifest as qualitative improvements observed by a consumer upon use. The results are set forth in Table 2B.

TABLE 2B

| | | Inventive | | | | | | | |
|---|---|---|---|---|---|---|---|---|---|
| | | Ratio | | | | | | | |
| | | 2:1 | 1:1 | 1:2 | 1:3 | 1:5 | 1:10 | Comparative | |
| Time | Property | 2A | 2B | 2C | 2D | 2E | 2F | C8 | C9 |
| A | Elongation | 4.5 | 4.5 | 4.5 | 4.5 | 4.5 | 4.5 | 4.5 | 1 |
| B | Stiffness | 2 | 3 | 3 | 3 | 3.5 | 5 | 2 | 1 |
| | Elongation | 2 | 3 | 3.5 | 3 | 3.5 | 4 | 2 | 1 |
| | Frizz | 2 | 1 | 3 | 3 | 2.5 | 3 | 2 | 1 |

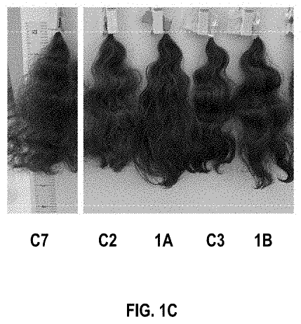

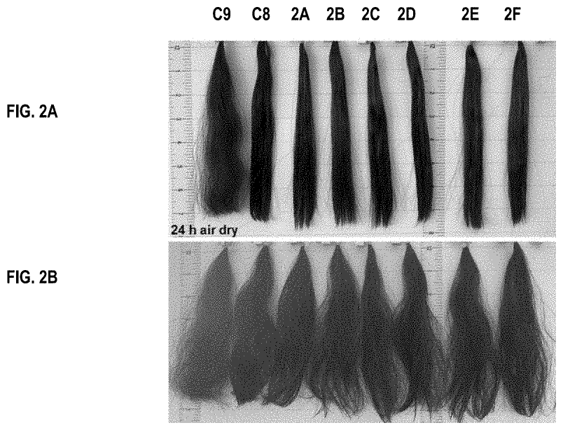

As can be seen in Table 2B, each of compositions 2A-2F and C8 provide good elongation benefits to curly hair after 24 hours (FIG. 2A), compared to composition C9. However, after exposure to high humidity conditions, inventive compositions 2A-2F demonstrate lastingness of elongation and anti-frizz benefits (FIG. 2B). Although composition 2F demonstrated the most stiffness of the swatch, the hair was not considered to be unacceptably stiff. These results are considered to be statistically significant, with the inventive compositions showing significant outperformance of the comparative compositions.

### Example 2-2—Very Curly Hair

The same study was repeated on swatches of very curly hair. The same results as shown above in Table 2B and FIGS. 2A-2B were observed.

The results in Examples 2-1 and 2-2 demonstrate that the surprisingly synergistic benefits are achieved with compositions according to the disclosure across a wide range of ratios of polysaccharide thickening agent to inorganic phosphate.

### Example 3—Compositions With Various Inorganic Phosphates

The following Examples 3-1 to 3-4 demonstrate the surprising and unexpected beneficial effects achieved using various inorganic phosphates in compositions according to the disclosure.

### Example 3-1—Curly Hair

The inventive and comparative compositions in Table 3A were prepared.

TABLE 3A

| | Inventive | | Comparative | | | |
|---|---|---|---|---|---|---|
| | 3A | 3B | C11 | C12 | C13 | C14 |
| Xanthan gum | 1 | 1 | | 1 | | |
| Sodium hexa- | 3 | | | | 3 | |
| metaphosphate | | | | | | |
| Sodium mono- | | 3 | | | | 3 |
| phosphate | | | | | | |
| Water | QS to | QS to | QS to | QS to | QS to | QS to |
| | 100 | 100 | 100 | 100 | 100 | 100 |

Six swatches of curly hair were washed with a standard shampoo and rinsed, and then equal amounts of compositions 3A-3B or C11-C14 were applied to separate wet hair swatches. The treated hair swatches were then brushed 5 times and allowed to air dry for 24 hours. The film on the hair was subsequently broken and the swatches were evaluated. The swatches were then put into a humidity chamber at 80% relative humidity at 25° C. and again evaluated.

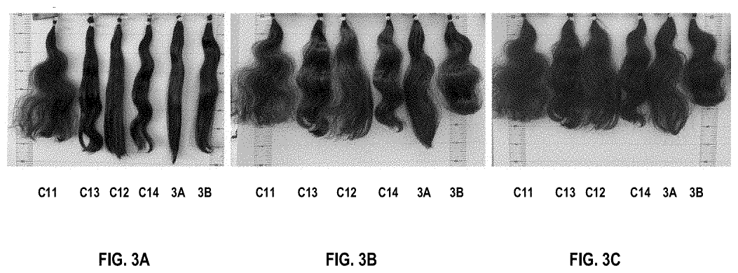

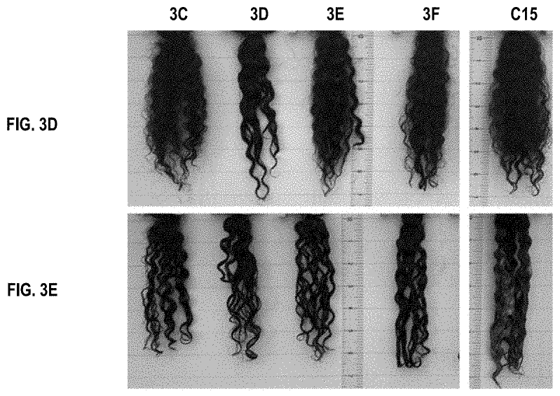

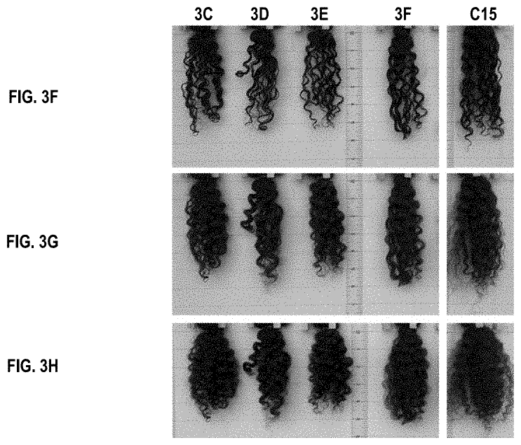

The hair swatches treated with each of compositions 3A-3B and C11-C14 were evaluated once the film was broken after drying for 24 hours (FIG. 3A), after 1 hour in the humidity chamber (FIG. 3B), and after 8 hours in the humidity chamber (FIG. 3C).

As can be seen in FIG. 3A, composition 3A provides the most elongation with frizz control after 24 hours. This benefit lasts even after 1 hour and 8 hours in high humidity, as seen in FIGS. 3B-3C. Further, composition 3B provides the greatest degree of curl definition and frizz control after 1 hour and 8 hours in high humidity, as also seen in FIGS. 3B-3C.

### Example 3-2—Curly Hair

The same study was repeated on swatches of hair with a slightly greater degree of curl, and the same results as described in Example 3-1 and shown in FIGS. 3A-3C were observed.

### Example 3-3—Very Curly Hair

The same study was repeated again on swatches of very curly hair, and the same results as described in Example 3-1 and shown in FIGS. 3A-3C were again observed.

### Example 3-4—Very Curly Hair

The inventive and comparative compositions in Table 3B were prepared.

TABLE 3B

| | Inventive | | | | Comp. |
|---|---|---|---|---|---|
| | 3C | 3D | 3E | 3F | C15 |
| Polyols (hexylene glycol | 2.0 | 2.0 | 2.0 | 2.0 | 2.0 |
| and capryl glycol) | | | | | |
| Xanthan gum | 0.75 | 0.75 | 0.75 | 0.75 | 0.75 |
| VP/Methacrylamide/Vinyl | 0.20 | 0.20 | 0.20 | 0.20 | 0.20 |
| Imidazole copolymer | | | | | |
| Hydroxypropyl guar | 0.20 | 0.20 | 0.20 | 0.20 | 0.20 |
| Sodium C14-C16 olefin | 0.19 | 0.19 | 0.19 | 0.19 | 0.19 |
| sulfonate | | | | | |
| Ethylhexylglycerin | 0.90 | 0.90 | 0.90 | 0.90 | 0.90 |
| Additives (preservatives, | <1 | <1 | <1 | <1 | <1 |
| vitamins, fragrance, pH | | | | | |
| adjusters/buffers) | | | | | |
| Solvent (water + non- | QS to | QS to | QS to | QS to | QS to |
| aqueous solvents) | 100 | 100 | 100 | 100 | 100 |
| *Tetrasodium etidronate | 2.0 | | | | |
| *Tetrasodium | | 2.0 | | | |
| pyrophosphate | | | | | |
| *Etidronic acid | | | 2.0 | | |
| *Sodium | | | | 2.0 | |
| hexametaphosphate | | | | | |

*These components were added as a super-addition, i.e. the ingredients listed above already added to 100%, before the various phosphates were added.

Five swatches of very curly hair were washed with a standard shampoo and rinsed (FIG. 3D). Equal amounts of compositions 3C-3F or C15 were applied to separate wet hair swatches and the treated hair swatches were detangled (FIG. 3E). The swatches were then allowed to air dry for 16 hours (FIG. 3F). The film on the hair was subsequently broken with three passes of the hand/fingers (FIG. 3G). The swatches were then put into a humidity chamber at 80% relative humidity at 25° C. and evaluated after 1 hour (FIG. 3H).

As can be seen in FIGS. 3D-3H, at every time point after the compositions were applied to the hair swatches, the hair treated with composition 3F showed the greatest degree of elongation, whereas the hair treated with compositions 3C-3E showed the greatest degree of curl definition. All of the hair treated with compositions 3C-3F showed significantly better frizz control than the hair treated with composition C15.

The results in Examples 3-1 to 3-4 therefore demonstrate the synergistic benefits of lasting frizz control, curl elongation, and/or curl definition provided by the combination of inorganic phosphates and polysaccharide thickening agents according to the disclosure.

### Example 4—pH Effect

The inventive and comparative compositions in Table 4 were prepared. The pH of compositions 4A-4E was determined to be 6.33, and subsequently the pH of each of compositions 4A and 4C-4E was adjusted using citric acid or sodium hydroxide, as needed to achieve the final pH.

TABLE 4

| | Inventive | | | | | Comparative | |
|---|---|---|---|---|---|---|---|
| | 4A | 4B | 4C | 4D | 4E | C16 | C17 |
| Xanthan gum | 1 | 1 | 1 | 1 | 1 | 1 | |
| Sodium hexa- | 3 | 3 | 3 | 3 | 3 | | |
| metaphosphate | | | | | | | |
| Water | QS to | QS to | QS to | QS to | QS to | QS to | QS to |
| | 100 | 100 | 100 | 100 | 100 | 100 | 100 |
| Initial pH | 6.33 | 6.33 | 6.33 | 6.33 | 6.33 | | |
| Citric acid | QS | | | | | | |
| Sodium | | | QS | QS | QS | | |
| hydroxide | | | | | | | |
| Final pH | 4.75 | 6.33 | 8.52 | 10.0 | 12.12 | | |

### Example 4-1—Curly Hair

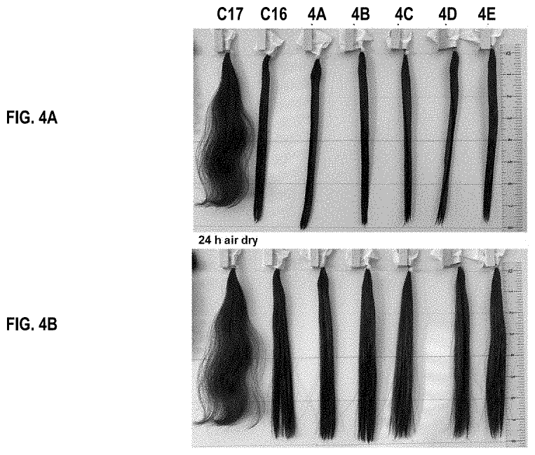

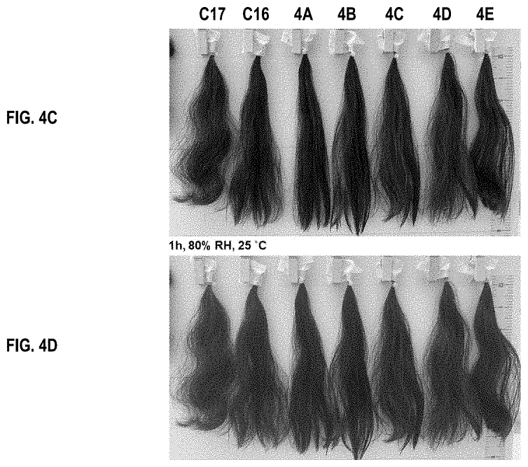

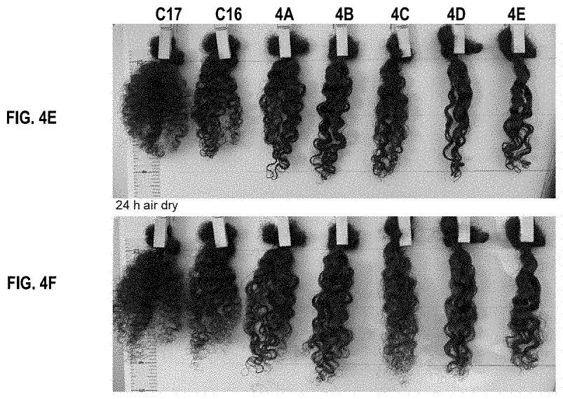

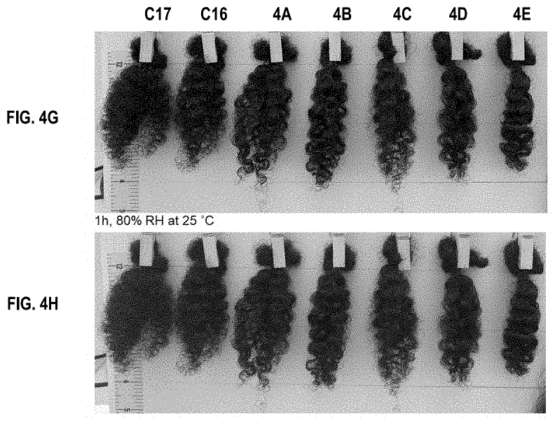

Seven swatches of curly hair were washed with a standard shampoo and rinsed, and then equal amounts of compositions 4A-4E or C16-C17 were applied to separate wet hair swatches. The treated hair swatches were then brushed 5 times and allowed to air dry for 24 hours (FIG. 4A). The film on the hair was subsequently broken and the swatches were evaluated for softness, elongation, and frizz (FIG. 4B). The swatches were then put into a humidity chamber at 80% relative humidity at 25° C. and again evaluated for elongation and frizz after 1 hour (FIG. 4C) and 8 hours (FIG. 4D).

Each of compositions 4A-4E and C16 provided softness to the hair, with compositions 4B-4E providing the most softness.

As can be seen in FIGS. 4A-4B, each of compositions 4A-4E provided good elongation and frizz control to curly hair before exposure to high humidity, compared to compositions C16 and C17. As seen in FIGS. 4C-4D, these elongation and anti-frizz benefits lasted even after 1 hour and 8 hours at high humidity.

### Example 4-2—Very Curly Hair

The same study was repeated on swatches of very curly hair. Seven swatches of very curly hair were washed with a standard shampoo and rinsed, and then equal amounts of compositions 4A-4E or C16-C17 were applied to separate wet hair swatches. The treated hair swatches were then detangled and allowed to air dry for 24 hours (FIG. 4E). The film on the hair was subsequently broken and the swatches were evaluated for softness, elongation, and frizz (FIG. 4F). The swatches were then put into a humidity chamber at 80% relative humidity at 25° C. and again evaluated for elongation and frizz after 1 hour (FIG. 4G) and 8 hours (FIG. 4H).

As can be seen in FIGS. 4E-4F, each of compositions 4A-4E provided good elongation and frizz control to very curly hair before exposure to high humidity, compared to compositions C16 and C17. As seen in FIGS. 4G-4H, these elongation and anti-frizz benefits lasted even after 1 hour and 8 hours at high humidity.

The results in Examples 4-1 and 4-2 demonstrate that good elongation and frizz control is provided by the combination of inorganic phosphate and polysaccharide thickening agent having a range of pH values.

### Example 5—Compositions With Addition of Various Polvols

The inventive and comparative compositions in Table 5A were prepared.

TABLE 5A

| | Inventive | | | | | | | Comp. |
|---|---|---|---|---|---|---|---|---|
| | 5A | 5B | 5C | 5D | 5E | 5F | 5G | C18 |
| Xanthan | 1 | 1 | 1 | 1 | 1 | 1 | 1 | |
| gum | | | | | | | | |
| Sodium | 3 | 3 | 3 | 3 | 3 | 3 | 3 | |
| hexameta- | | | | | | | | |
| phosphate | | | | | | | | |
| Glycerin | | 1 | 3 | 5 | | | | |
| Hexylene | | | | | 1 | 3 | 5 | |
| glycol | | | | | | | | |
| Water | QS to | QS to | QS to | QS to | QS to | QS to | QS to | QS to |
| | 100 | 100 | 100 | 100 | 100 | 100 | 100 | 100 |

Eight swatches of curly or very curly hair were washed with a standard shampoo and rinsed, and then equal amounts of compositions 5A-5G or C18 were applied to separate wet hair swatches. The treated curly hair swatches were then brushed 5 times and allowed to air dry for 24 hours. The treated very curly hair swatches were then detangled by hand using the fingers. The film on the hair was subsequently broken with the number of passes of the hand/fingers shown in Table 5B, and the swatches were put into a humidity chamber at 80% relative humidity at 25° C. and evaluated for elongation and frizz after 8 hours.

The elongation, frizz, and softness of each swatch was assigned a rating from 1 to 5, with a rating of 1 meaning that the swatch had the most frizz and least elongation, and a rating of 5 meaning that the swatch had the least frizz and most elongation. Such an evaluation is how properties imparted by such compositions are typically evaluated in the art, since beneficial properties of new formulations generally manifest as qualitative improvements observed by a consumer upon use. The results are set forth in Table 5B.

TABLE 5B

| | Inventive | | | | | | | Comp. |
|---|---|---|---|---|---|---|---|---|
| | 5A | 5B | 5C | 5D | 5E | 5F | 5G | C18 |
| Film passes | 3 | 2 | 2 | 2 | 2 | 2 | 2 | 0 |
| Frizz | 3 | 3 | 3 | 3 | 3 | 2.5 | 1.5 | 1 |
| Elongation | 4 | 3.5 | 1.75 | 1.75 | 4 | 2 | 1 | 1 |

As seen in Table 5B, hair swatches treated with compositions containing a polyol demonstrated improved elongation and/or anti-frizz effects than hair swatches treated with composition C18. Hair swatches treated with compositions 5B-5G were noticeably softer than hair swatches treated with composition 5A or C18. These results are considered to be statistically significant, with the inventive compositions showing significant outperformance of the comparative composition.

This Example demonstrates that the addition of a polyol in compositions according to the disclosure imparts softness benefits to the hair, while maintaining other benefits such as elongation and/or anti-frizz properties.

The above examples demonstrate that compositions according to the disclosure comprising at least one inorganic phosphate and at least one anionic or nonionic polysaccharide thickening agent provide surprising and unexpected synergistic benefits such as curl definition, curl elongation, and/or frizz control to hair.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1-63. (canceled)

64. A composition comprising:
(a) at least one inorganic phosphate or salt thereof,
(b) at least one polysaccharide thickening agent chosen from anionic and/or nonionic polysaccharide thickening agents, and
(c) at least one solvent,
wherein the total amount of (a) inorganic phosphates and salts thereof ranges from about 0.1% to about 20%;
wherein the total amount of (b) anionic and nonionic polysaccharide thickening agents ranges from about 0.1% to about 10%; and
wherein all amounts are by weight, relative to the total weight of the composition.

65. The composition of claim 64, having a weight ratio of (b) the at least one polysaccharide thickening agent to (a) the at least one inorganic phosphate or salt thereof ranging from about 2:1 to about 1:10.

66. The composition of claim 64, wherein the at least one inorganic phosphate comprises at least one inorganic polyphosphate.

67. The composition of claim 66, wherein the at least one inorganic polyphosphate is chosen from tetrasodium etidronate, tetrasodium pyrophosphate, etidronic acid, sodium hexametaphosphate, or mixtures thereof.

68. The composition of claim 64, further comprising at least one lactone, wherein the total amount of lactone(s) ranges from about 0.1% to about 10% by weight, relative to the total weight of the composition.

69. The composition of claim 64, wherein the at least one polysaccharide thickening agent is chosen from hydroxypropyl guar, carrageenan, xanthan gum, cellulose gum, cyclodextrin, sclerotium gum, polyvinyl alcohol, or mixtures thereof.

70. The composition of claim 64, having a pH ranging from about 4 to about 12.5.

71. The composition of claim 64, further comprising at least one polyol, wherein the total amount of polyol(s) ranges from about 0.1% to about 10% by weight, relative to the total weight of the composition.

72. The composition of claim 64, further comprising at least one anionic surfactant, wherein the total amount of anionic surfactant(s) ranges from about 0.1% to about 15% by weight, relative to the total weight of the composition.

73. The composition of claim 64, wherein the composition is a leave-in composition.

74. A composition comprising:
(a) about 0.1% to about 20% of sodium hexametaphosphate,
(b) about 0.1% to about 10% of at least one polysaccharide thickening agent chosen from anionic and/or nonionic polysaccharide thickening agents, and
(c) at least one solvent,
wherein all amounts are by weight, relative to the total weight of the composition.

75. The composition of claim 74, having a weight ratio of (b) the at least one polysaccharide thickening agent to (a) sodium hexametaphosphate ranging from about 2:1 to about 1:10.

76. The composition of claim 74, further comprising at least one lactone, wherein the total amount of lactone(s) ranges from about 0.1% to about 10% by weight, relative to the total weight of the composition.

77. The composition of claim 76, having a weight ratio of lactone(s) to sodium hexametaphosphate ranging from about 5:1 to about 1:1.

78. The composition of claim 74, wherein the at least one polysaccharide thickening agent is chosen from hydroxypropyl guar, carrageenan, xanthan gum, cellulose gum, cyclodextrin, sclerotium gum, polyvinyl alcohol, or mixtures thereof.

79. The composition of claim 74, having a pH ranging from about 4 to about 12.5.

80. The composition of claim 74, further comprising at least one polyol, wherein the total amount of polyol(s) ranges from about 0.1% to about 10% by weight, relative to the total weight of the composition.

81. The composition of claim 74, further comprising at least one anionic surfactant, wherein the total amount of anionic surfactant(s) ranges from about 0.1% to about 15% by weight, relative to the total weight of the composition.

82. The composition of claim 74, wherein the composition is a leave-in composition.

83. A method for styling or shaping hair comprising applying to the hair a composition comprising:
(a) at least one inorganic phosphate or salt thereof,
(b) at least one polysaccharide thickening agent chosen from anionic and/or nonionic polysaccharide thickening agents, and
(c) at least one solvent,
wherein the total amount of (a) inorganic phosphates and salts thereof ranges from about 0.1% to about 20%;
wherein the total amount of (b) anionic and nonionic polysaccharide thickening agents ranges from about 0.1% to about 10%; and
wherein all amounts are by weight, relative to the total weight of the composition.
